(19) United States
(12) Patent Application Publication
(10) Pub. No.: US 2017/0239076 A1
(43) Pub. Date: Aug. 24, 2017

(54) WEARABLE DEVICE FOR SNORING

(71) Applicant: Merilou Stanton, Bradford, NH (US)

(72) Inventor: Merilou Stanton, Bradford, NH (US)

(21) Appl. No.: 15/331,794

(22) Filed: Oct. 21, 2016

Related U.S. Application Data
(60) Provisional application No. 62/298,915, filed on Feb. 23, 2016.

(51) Int. Cl. A61F 5/56; A61F 5/37

(52) U.S. Cl. CPC A61F 5/56; A61F 5/3707

(57) ABSTRACT

A wearable device for snoring including a head support and harness configured to retain the head support between the chin and the collar bone of the user-wearer when worn. The harness having an adjustable attachment mechanism for a variety of adjustment lengths around the neck of the user-wearer. The device provides a means by which to deter snoring and provide a more restful sleep while offering a variety of positions of the head with respect to the body.

### CROSS-REFERENCE TO RELATED APPLICATION

The present application is related to and claims priority from prior provisional application Ser. No. 62/298,915, filed Feb. 23, 2016 which application is incorporated herein by reference in its entirety.

### BACKGROUND OF THE INVENTION

The following includes information that may be useful in understanding the present disclosure. It is not an admission that any of the information provided herein is prior art, or material, to the presently described or claimed inventions, or that any publication or document that is specifically or implicitly referenced is prior art.

1. Field of the Invention

The present disclosure relates generally to the field of anti-snoring or mouth closing devices and more specifically relates to devices for preventing snoring.

2. Description of Related Art

Generally speaking sleep is a naturally recurring state of mind characterized by altered consciousness, relatively inhibited sensory activity, inhibition of nearly all voluntary muscles, and reduced interactions with surroundings. It is distinguished from wakefulness by a decreased ability to react to stimuli, but is more easily reversed than the state of hibernation or of being comatose. Mammalian sleep occurs in repeating periods, in which the body alternates between two highly distinct modes known as non-REM and REM sleep. REM stands for “rapid eye movement” but involves many other aspects including virtual paralysis of the body. Many people have problems sleeping.

At rest, many people are prone to dropping their jaw and breathing through their open mouth, which can cause snoring, restless sleep, and possibly even severe medical conditions, like sleep apnea. Some people may wear various chinstraps currently on the market to hold their mouth shut, but such devices can be uncomfortable and don't provide enough support to keep the mouth entirely closed. A suitable solution is desired.

Several attempts have been made to solve the above-mentioned problems such as those found in U.S. Pat. No. 7,789,843 to Ray, U.S. Pat. No. 5,289,829 to Roehrig, U.S. Pat. No. 6,171,314 to Rotramel, U.S. Pat. No. 6,969,366 to Reddick, and U.S. Pat. No. 8,733,362 to Krook et al. This art is representative of anti-snoring or mouth closing devices. However, none of the above references, taken either singly or in combination, is seen to describe the invention as claimed.

### BRIEF SUMMARY OF THE INVENTION

In view of the foregoing disadvantages inherent in the known anti-snoring or mouth closing devices art, the present disclosure provides a novel wearable device for snoring. The general purpose of the present disclosure, which will be described subsequently in greater detail is to provide a means by which to mitigate snoring and provide more restful sleep. The present disclosure provides chin support for people whose mouth opens during sleep and comfortably holds up the chin, regardless the sleeping position. The device promotes breathing through the nose, and helps reduce snoring. It replaces the use of existing chinstraps, which are uncomfortable and ineffective and shaped so as to limit positions of the head in relation to the body. As such, the present disclosure may help users sleep more soundly as well as offer more options for positions of the head in relation to the body.

A wearable device for snoring is disclosed herein, including a head support including a jawbone interface and a neck spacer. The wearable device for snoring further includes a harness configured to retain the head support between the chin and the collar bone of a user-wearer when and the harness is coupled to the head support. The harness includes an adjustable strap and toolless fastener, such that the adjustable strap and toolless fastener are configured to fasten around a neck of the user-wearer, and further configured to hold the head support in place between the neck and collar bone proximate a throat of the user-wearer when worn.

According to another embodiment, a method for using wearable device for snoring is also disclosed herein. The method for providing a means by which to deter snoring includes the steps of: placing the first neck spacer between a chin and a neck of a user-wearer, securing a fastener band around the neck, and sleeping with the wearable device. The method may further include the steps of: removing the wearable device from the user-wearer, removing the first neck spacer from the wearable device, replacing the first neck spacer with a second neck spacer, the second neck spacer having a different size than the first neck spacer, and washing an outer covering of the wearable device.

For purposes of summarizing the disclosure, certain aspects, advantages, and novel features of the disclosure have been described herein. It is to be understood that not necessarily all such advantages may be achieved in accordance with any one particular embodiment of the disclosure. Thus, the disclosure may be embodied or carried out in a manner that achieves or optimizes one advantage or group of advantages as taught herein without necessarily achieving other advantages as may be taught or suggested herein. The features of the disclosure which are believed to be novel are particularly pointed out and distinctly claimed in the concluding portion of the specification. These and other features, aspects, and advantages of the present disclosure will become better understood with reference to the following drawings and detailed description.

### BRIEF DESCRIPTION OF THE DRAWINGS

The figures which accompany the written portion of this specification illustrate embodiments and methods of use for the present disclosure, directed towards a wearable device for snoring, which is constructed and operative according to the teachings of the present disclosure.

FIG. 1 shows a perspective view, illustrating a wearable device for snoring during an ‘in-use’ condition, according to an embodiment of the disclosure.

FIG. 2 is an alternate view, illustrating the during an ‘in-use’ condition, according to an embodiment of the disclosure.

FIG. 3 is a perspective view, illustrating head support and harness assembly, according to an embodiment of the present disclosure.

FIG. 4 is an exploded view of a head support and harness assembly, according to an embodiment of the present disclosure.

FIG. 5 is a flow diagram illustrating a method of use for wearable device for snoring according to an embodiment of the present disclosure.

The various embodiments of the present disclosure will hereinafter be described in conjunction with the appended drawings, wherein like designations denote like elements.

### DETAILED DESCRIPTION

As discussed above, embodiments of the present disclosure relate to an anti-snoring or mouth closing devices and more particularly to a wearable device for snoring as used to improve means by which to deter snoring and provide more restful sleep.

The present disclosure holds significant improvements and serves as a wearable device for snoring. Preferably, an anti-snoring or mouth closing devices should provide a means by which to deter snoring and enhance restful sleep and, yet would operate reliably and be manufactured at a modest expense. Thus, a need exists for a reliable wearable device for snoring to avoid the above-mentioned problems.

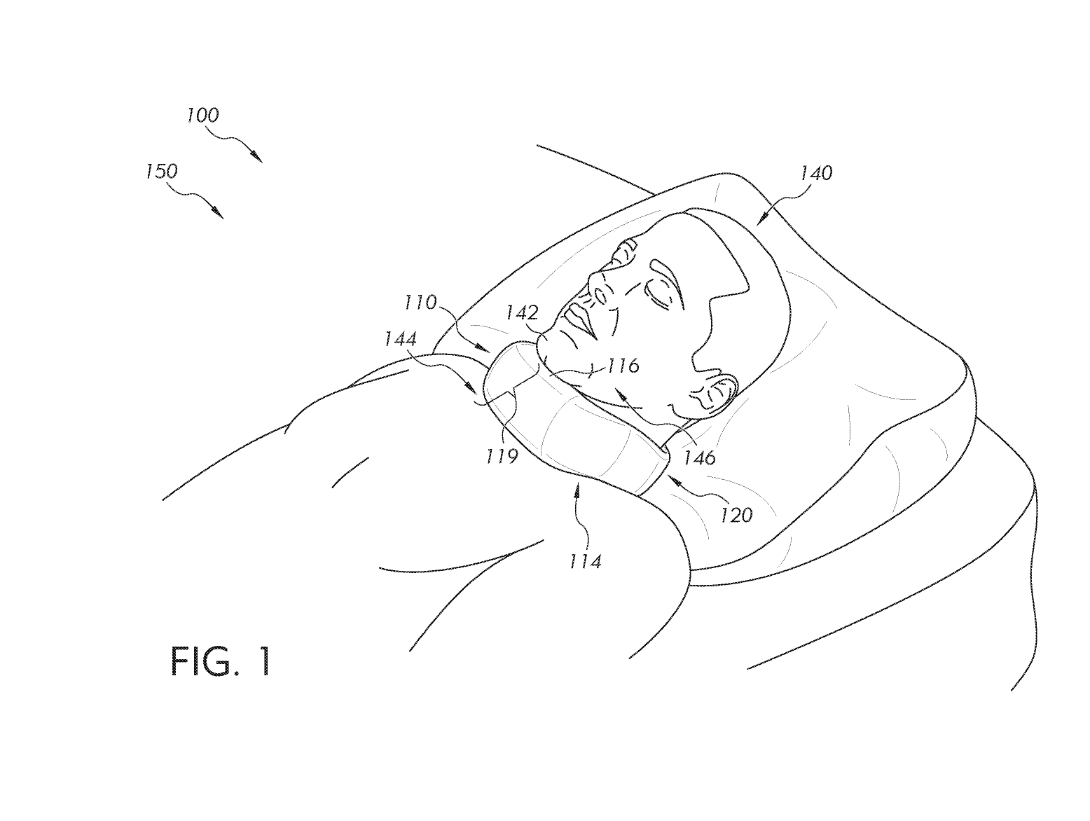

Referring now more specifically to the drawings by numerals of reference there is shown in FIGS. 1-4, various views of a wearable device for snoring 100. The wearable device for snoring 100 may include a head support 110 and harness 120. The head support 110 may include a neck spacer 114 and a jawbone interface 116. The harness 120 may include an adjustable strap 124 and a toolless fastener 126. The toolless fastener may include a hook-and-loop fastener mechanism and the harness maybe fastened about a circumference of 15 inches.

As discussed further below, the wearable device for snoring 100 may further include an outer cover made of a fabric material, the outer cover encasing a head support. The outer cover may be pleated, and the outer cover and the neck spacer may be flexible together about the neck. The fabric material may be made of a washable material. The outer cover may include an access opening through which the neck spacer may be removed from and inserted into the outer cover. The access opening may include a hook and loop fastener configured to close the access opening. The jawbone interface and the neck spacer may be combined together as an elongated cushioning material having length along an elongation axis and a thickness perpendicular to said elongation axis, the thickness being at least a minimum gap. The elongated cushioning material may be insertable into and removable from the outer cover. The is adjustable between a plurality of neck sizes. The wearable device may further include a set of instructions and be arranged as a kit.

FIG. 1 shows the wearable device for snoring 100 in an ‘in-use’ condition 150. Here, the wearable device for snoring 100 includes the head support 110 and the harness 120, where the harness 120 is coupled to the head support 110.

As above, the head support 110 includes the neck spacer 114 and the jawbone interface 116. More particularly, the jawbone interface 116 is shown configured to receive and support at least a portion of a jawbone 146 of a user-wearer 140, and the neck spacer 114 is shown configured to maintain a minimum gap 119 between a chin 142 and a collar bone 144 of the user-wearer 140. According to one embodiment, the minimum gap 119 may be at least 2 inches.

Also as above, the harness 120 includes the adjustable strap 124 and the toolless fastener 126. More particularly the adjustable strap 124 is shown configured to fasten around a neck of the user-wearer 140 and further configured to hold the head support 110 in place between the neck and the collar bone 144 proximate to a throat of the user-wearer 140 when worn.

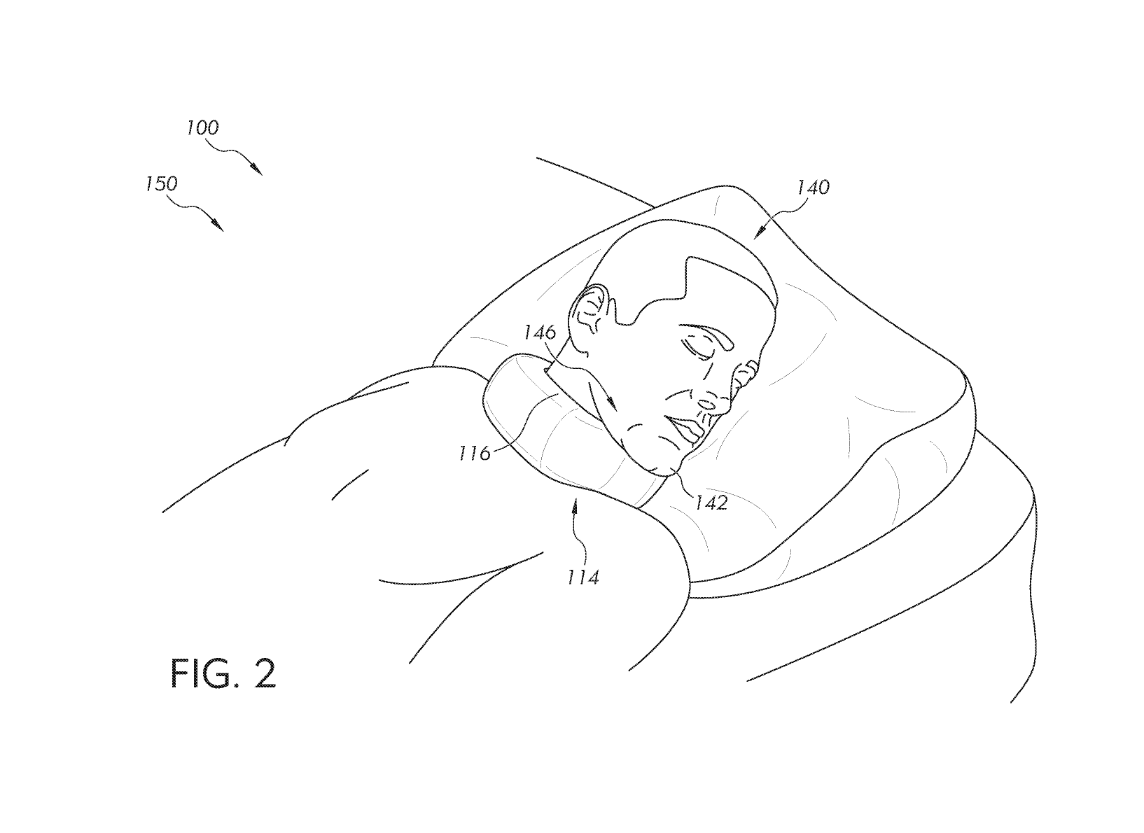

Referring now to FIG. 2, showing an ‘in-use’ condition 150, with the wearable device for snoring 100 in a preferred embodiment. As discussed below, the jawbone interface 116 and the neck spacer 114 are shown combined together and extending around the front half of the neck of the user-wearer 140. This configuration provides the advantage of multiple head positions for the user-wearer 140 during periods of sleep.

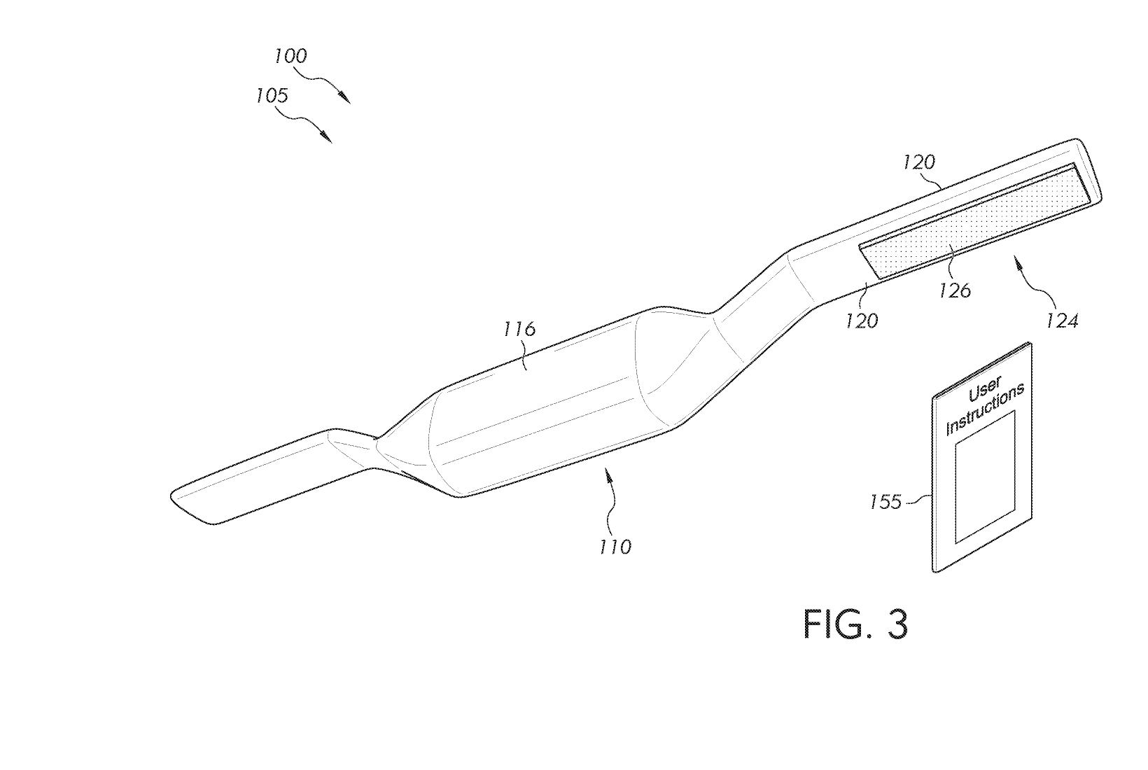

Referring now to FIG. 3, showing a bottom perspective view of the wearable device for snoring 100 in a free state. As above, the wearable device 100 is shown including the head support 110 and the harness 120 coupled together as a single unit. According to one embodiment, the harness 120 may include an adjustable strap 124 and toolless fastener 126. As shown, the harness 120 is adjustable among a plurality of neck sizes, corresponding to the adjustability of the adjustable strap 124. According to one embodiment, the harness 120 is adjustable to provide a space of 0.5 inch between the head support 110 and the neck of the user-wearer 140 (e.g., for comfort, for breathing, for swallowing, etc.). In one preferred embodiment, the toolless fastener 126 may include a hook-and-loop attachment mechanism further offering the ability to adjust to a large variety of sizes. As shown, the adjustability an adjustable strap 124, and corresponding neck sizes, will vary with the length of the toolless fastener 126 (e.g., the length of hook-and-loop attachment material fixed to the adjustable strap 124). In alternate embodiments, the toolless fastener 126 may include at least one button fastener or at least one snap fastener configured to couple the adjustable strap 124 about different size necks.

According to one embodiment, the wearable device 100 may be arranged as a kit 105. In particular, the wearable device 100 may further include a set of instructions 155. The instructions 155 may detail functional relationships in relation to the structure of the wearable device 100 (such that wearable device 100 can be used, maintained, or the like, in a preferred manner).

It is understood the wearable device 100 may be manufactured and provided for sale in a wide variety of sizes and shapes for a wide assortment of applications. Upon reading this disclosure, it should be appreciated that, under appropriate circumstances, considering such issues as design preference, user preferences, marketing preferences, cost, structural requirements, available materials, technological advances, etc., other kit contents or arrangements are contemplated (e.g., more or less named components, customized parts, different color combinations, individual parts provided separately, etc.).

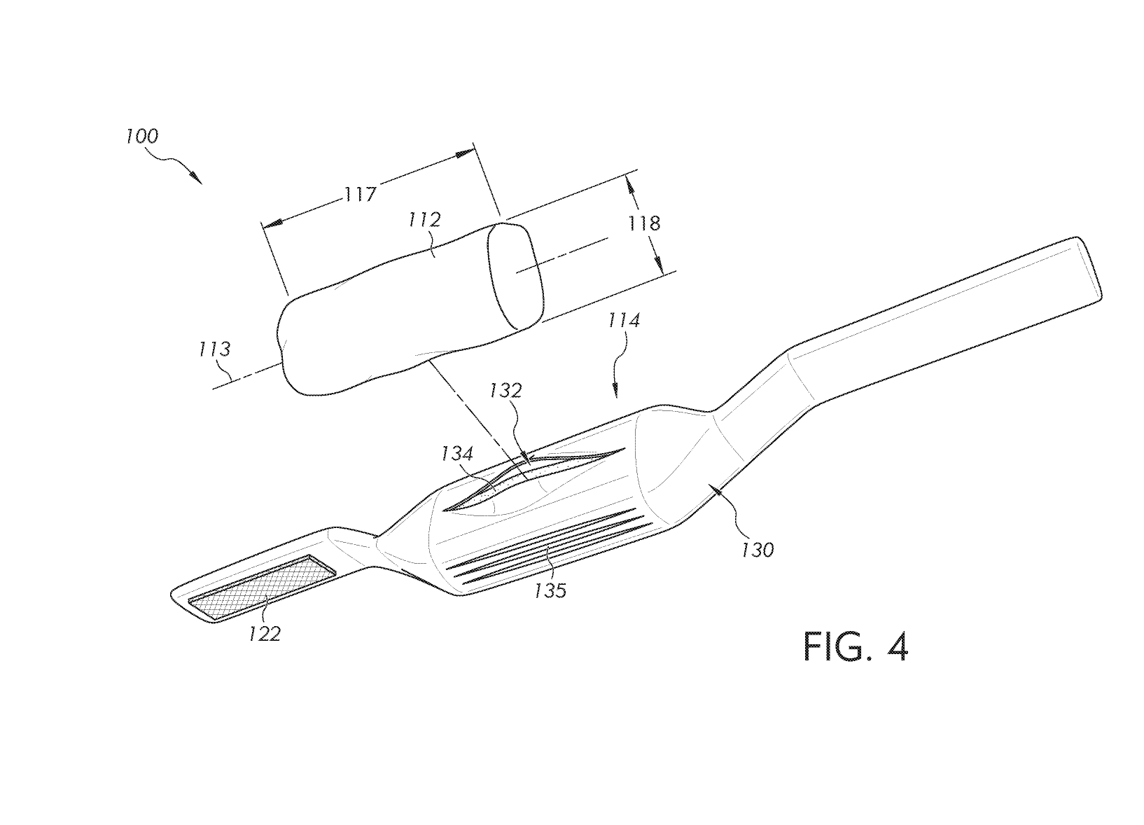

Referring now to FIG. 4, showing an exploded view of the wearable device for snoring 100, additional features are shown. As above, the jawbone interface 116 and neck spacer 114 are combined as a single elongated cushioning material 112 having an outer cover 130. The elongated cushioning material 112 may have length 117 along an elongation axis 113 and a thickness 118 measured perpendicular to the elongation axis 113. It is understood that, as a cushioning material, the thickness 118 of the elongated cushioning material 112 will necessarily vary depending on how much compression is applied by the user-wearer. Notwithstanding, the thickness 118 may be at least the minimum gap 119 (FIG. 1). Alternately, the thickness 118 may be selected such that the jawbone interface 116 and neck spacer 114 arrive at the minimum gap 119 when compressed under normal pressures. Here, the combined jawbone interface 116 and neck spacer 114 is shown generally having a constant thickness. In alternate embodiments, one or both of the jawbone interface 116 and neck spacer 114 may have varying thicknesses along the elongation axis 113.

The outer cover 130 includes an access opening 132 configured to provide access such that the elongated cushioning material 112 is insertable into and removable from the outer cover 130. This may be beneficial for cleaning and hygiene in general. According to one embodiment, the outer cover 130 may be expandable (e.g., pleated 135) or otherwise configured to accommodate various lengths and thicknesses of the elongated cushioning material 112. Outer cover 130 and neck spacer 114 are flexible together about the neck. The fabric material of outer cover 130 is made of a washable material. In a preferred embodiment, the access opening 132 may include a hook-and-loop fastener 134 arrangement configured to close the access opening 132. In alternate preferred embodiments, access opening 132 may include at least one button fastener or at least one snap fastener configured to close the access opening 132.

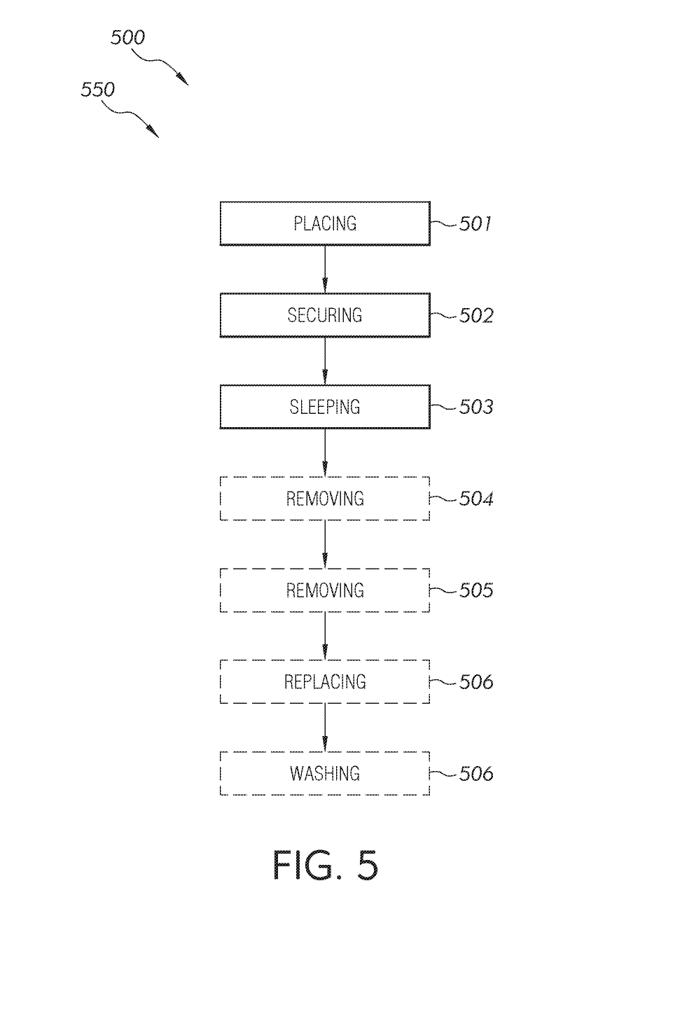

Referring now to FIG. 5 showing a flow diagram illustrating a method of use 500 for the wearable device for snoring 100 according to the present disclosure and FIGS. 1-4. As shown, method of use 500 may include the steps of: step one 501, placing the first neck spacer between a chin and a neck of a user-wearer; step two 502, securing a harness around the neck; and step three 503, sleeping with the wearable device for snoring. The method of use 500 may also include the steps of: step four 504 removing the wearable device for snoring from the user-wearer; step five 505 removing the first neck spacer from the wearable device for snoring; step six, 506, replacing the first neck spacer with a second neck spacer, the second neck spacer having a different size than the first neck spacer; and step seven, 507 washing an outer cover of the wearable device for snoring.

It should be noted that steps 504, 505, 506, and 507 are optional steps and might not be implemented in all cases. Optional steps of method of use 500 are illustrated using dotted lines in FIG. 5 so as to distinguish them from the other steps of method of use 500. It should be noted that the steps described in the method of use can be carried out in many different orders according to user preference. The use of “step of” should not be interpreted as “step for”, in the claims herein and is not intended to invoke the provisions of 35 U.S.C. §112(f). Upon reading this specification, it should be appreciated that, under appropriate circumstances, considering such issues as design preference, user preferences, marketing preferences, cost, structural requirements, available materials, technological advances, etc., other methods of use arrangements such as, for example, different orders within above-mentioned list, elimination or addition of certain steps, including or excluding certain maintenance steps, etc., may be sufficient.

The embodiments of the disclosure described herein are exemplary and numerous modifications, variations and rearrangements can be readily envisioned to achieve substantially equivalent results, all of which are intended to be embraced within the spirit and scope of the disclosure. Further, the purpose of the foregoing abstract is to enable the U.S. Patent and Trademark Office and the public generally, and especially the scientist, engineers and practitioners in the art who are not familiar with patent or legal terms or phraseology, to determine quickly from a cursory inspection the nature and essence of the technical disclosure of the application.

Those with ordinary skill in the art will now appreciate that upon reading this specification and by their understanding the art of anti-snoring or mouth closing devices as described herein, methods of devices for preventing snoring will be understood by those knowledgeable in such art.

What is claimed is new and desired to be protected by Letters Patent is set forth in the appended claims:

1. A wearable device for snoring comprising:
a head support including a jawbone interface and a neck spacer, said jawbone interface configured to receive and support at least a portion of a jawbone of a user-wearer, and said neck spacer configured to maintain a minimum gap between a chin of said user-wearer and a collar-bone of said user-wearer; and
a harness configured to retain the head support between the chin and the collar bone of the user-wearer, when worn.

2. The wearable device of claim 1, wherein the minimum gap is at least 2 inches; and
wherein the harness is coupled to the head support, the harness including an adjustable strap and toolless fastener, the adjustable strap and toolless fastener configured to fasten around a neck of the user-wearer, and further configured to hold the head support in place between the neck and the collar bone proximate a throat of the user-wearer, when worn.

3. The wearable device of claim 2, wherein the toolless fastener includes a hook-and-loop fastener mechanism.

4. The wearable device of claim 2, wherein the harness maybe fastened about a circumference of 15 inches.

5. The wearable device of claim 1, further comprising an outer cover made of a fabric material, the outer cover encasing the head support.

6. The wearable device of claim 5, wherein said outer cover is pleated, and the outer cover and the neck spacer are flexible together about the neck.

7. The wearable device of claim 5, wherein the fabric material is made of a washable material.

8. The wearable device of claim 5, wherein said outer cover includes an access opening through which the neck spacer may be removed from and inserted into the outer cover.

9. The wearable device of claim 8, wherein the access opening includes a zipper configured to close said access opening.

10. The wearable device of claim 8, wherein the access opening includes a hook and loop fastener configured to close said access opening.

11. The wearable device of claim 8, wherein the access opening includes at least one button fastener configured to close said access opening.

12. The wearable device of claim 8, wherein the access opening includes at least one snap fastener configured to close said access opening.

13. The wearable device of claim 5, wherein the jawbone interface and the neck spacer are combined together as an elongated cushioning material having length along an elongation axis and a thickness perpendicular to said elongation axis, the thickness being at least the minimum gap, the elongated cushioning material insertable into and removable from the outer cover.

14. The wearable device of claim 13, wherein the elongated cushioning material includes a varying thickness along the elongation axis.

15. The wearable device of claim 2, wherein the harness is adjustable between a plurality of neck sizes.

16. The wearable device of claim 15, wherein the harness is further adjustable to provide a space of 0.5 inch between the head support and the neck of the user-wearer.

17. A wearable device for snoring comprising:
a head support including a jawbone interface and a neck spacer, said jawbone interface configured to receive and support at least a portion of a jawbone of a user-wearer, and said neck spacer configured to maintain a minimum gap between a chin of said user-wearer and a collar-bone of said user-wearer;
a harness configured to retain the head support between the chin and the collar bone of the user-wearer; when worn; and
an outer cover made of a fabric material, the outer cover encasing the head support;
wherein the minimum gap is at least 2 inches;
wherein the harness is coupled to the head support, the harness including an adjustable strap and toolless fastener, the adjustable strap and toolless fastener configured to fasten around a neck of the user-wearer, and further configured to hold the head support in place between the neck and the collar bone proximate a throat of the user-wearer when worn;
wherein the toolless fastener includes a hook-and-loop fastener mechanism;
wherein the harness maybe fastened about a circumference of 15 inches;
wherein said outer cover is pleated, and the outer cover and the neck spacer are flexible together about the neck;
wherein the fabric material is made of a washable material;
wherein the outer cover includes an access opening through which the neck spacer may be removed from and inserted into the outer cover;
wherein the access opening includes a hook and loop fastener configured to close said access opening;
wherein the jawbone interface and the neck spacer are combined together as an elongated cushioning material having length along an elongation axis and a thickness perpendicular to said elongation axis, the thickness being at least the minimum gap, the elongated cushioning material insertable into and removable from the outer cover;
wherein the harness is adjustable between a plurality of neck sizes; and
wherein the harness is further adjustable to provide a space of 0.5 inch between the head support and the neck of the user-wearer.

18. The wearable device of claim 17, further comprising a set of instructions; and
wherein the wearable device is arranged as a kit.

19. A method of using a wearable device for snoring, the wearable device including a first neck spacer coupled to a fastenable harness, the method comprising the steps of:
placing the first neck spacer between a chin and a neck of a user-wearer;
securing the fastenable harness the neck; and
sleeping with the wearable device.

20. The method of claim 19, further comprising the steps of:
removing the wearable device from the user-wearer;
removing the first neck spacer from the wearable device;
replacing the first neck spacer with a second neck spacer, the second neck spacer having a different size than the first neck spacer; and
washing an outer covering of the wearable device.
